(19) United States
(12) Patent Application Publication
(10) Pub. No.: US 2023/0111302 A1
(43) Pub. Date: Apr. 13, 2023

(54) MONOLITHIC MICROFLUIDIC ELECTROCHEMICAL SENSOR

(71) Applicant: The Board of Trustees of the University of Illinois, Urbana, IL (US)

(72) Inventors: Yurii VLASOV, Urbana, IL (US); Christopher Kenji BRENDEN, Urbana, IL (US)

(21) Appl. No.: 17/952,057

(22) Filed: Sep. 23, 2022

Related U.S. Application Data
(60) Provisional application No. 63/248,178, filed on Sep. 24, 2021.

(51) Int. Cl. A61B 5/145

(52) U.S. Cl. CPC A61B 5/14532; A61B 5/14546; G01N 33/5438

(57) ABSTRACT

Provided are electrochemical sensors for analyzing analytes. The sensors may comprise an implantable probe for analyzing a biological analyte, with those sensors described as electrochemical biosensors. Also provided are related methods of using and making the sensors. The electrochemical sensor is formed with an integrated on-chip probe body that provides for a buried microelectrode in a microfluidic channel etched in the probe body, such as a doped Si substrate. The fluidic system can, therefore, be quite small and suitable for in-vivo implantation and use, while withstanding high pressure. The fluidic system has specially-configured reagent channel to provide for periodic and convenient calibration, electrode cleaning and/or regeneration, without having to remove any sensor component from the implantation site.

### CROSS-REFERENCE TO RELATED APPLICATIONS

This application claims the benefit of priority to U.S. Provisional Pat. Application No. 63/248,178 filed Sep. 24, 2021, which is hereby incorporated by reference in its entirety.

### STATEMENT REGARDING FEDERALLY SPONSORED RESEARCH OR DEVELOPMENT

This invention was made with government support under NS107677 awarded by the National Institutes of Health. The government has certain rights in the invention.

### BACKGROUND OF INVENTION

Chemical sensors, including electrochemical sensors, are important in a range of applications where the detection and quantification of one or more materials is desired. Electrochemical sensors can have challenges related to long-term performance, particularly for applications where the environment that provides a sample to the sensor adversely impacts electrodes of the electrochemical sensor. This can be particularly challenging for in-vivo applications where the environment is such that the electrode fouling and degradation can be very rapid. That impact results in relatively short-lived sensors where, in order to increase sensor lifetime, time-consuming and biologically-impactful action is required.

There is a need in the art for electrochemical sensors having a long operational lifetime without an attendant need for time-consuming or active sensor manipulation that can disturb the surrounding environment to achieve long operational lifetime. Provided herein are electrochemical sensors, and related methods of making and using the sensors, that address this need by leveraging microfluidics and semiconductor manufacturing processes to obtain a reliable in-line electrochemical sensor useful for detection of a range of analytes for a range of applications.

### SUMMARY

Provided herein are electrochemical sensors having one or more electrodes that are integrated with an in-line microfluidic channel. In this manner, the electrodes are “buried” within microfluidic channels and are effectively protected from direct access with the surrounding environment that provides samples for analyte detection. This is particularly important for applications where the surrounding environment contains material that can result in degradation of sensor performance, such as by fouling agents that attack or interfere with electrode surfaces. The electrochemical sensors provided herein are further advantageously configured to have a reagent channel integrated with the electrode-containing microfluidic channel so that the electrodes can be regularly cleaned, calibrated and/or regenerated without having to actively manipulate the sensor. This is beneficial for applications where it is desired to minimize impact on the surrounding environment, such as biological tissues for electrochemical sensors that are biosensors. In this manner, insertion into biological systems is possible with minimal tissue disruption and adverse biological events. Analytes can be collected in vivo and over a time course, with high-resolution and sensitivity, both in a spatial sense (e.g., precise control over where the sample is obtained) and temporal (how the sample may change over time, including analytes within the sample).

The aforementioned advantages are achieved herein by a specially configured sensor having a small insertable footprint making the sensor suitable for in vivo implantation. The small footprint configured for insertion into biological tissue is obtained by an integrated fluidic component, where the channel and electrode(s) are integrated within the sensor body. This reflects that the sensor body is formed from a unitary material, including an undoped or a doped silicon substrate, and use of a plurality of different materials, including connecting multiple substrates to form a channel, is avoided. Depending on the application of interest, a doped, undoped, or patterned doped silicon substrate can be employed. The sensors and related methods provided herein are, of course, compatible with other types of applications, including in non-biological settings. For example, the sensing may be in an application associated with an industrial process, manufacturing process, quality control, water-quality application, such as in a sewage or treatment system used to monitor for materials of interest, including heavy metals.

Since the electrochemical sensor is small, the sample consumption amount can be minute, which provides a universal platform for trace detection circumstances, including in-vivo detection; compatible with various applications, including but not limited to neurochemical sampling. The sensor geometry can be configured to ensure minimal tissue damage, to achieve long-term monitoring for a range of applications. Examples include, glucose monitoring, blood analyte monitoring, vitamin level monitoring, hormone monitoring, cancer marker monitoring, disease state monitoring, including pathogen monitoring for bacterial or viral infection, and any of a range of chemicals that impact physiological function. Basically, any analyte that can be accessed by the distal tip end (e.g., the “sampling port”), introduced to the microfluidic channel, and is compatible for electrical detection by the electrodes, can be detected. This includes analytes associated with biological or non-biological applications.

Also provided herein are methods of using and methods making any of the electrochemical sensors described herein. In an embodiment, the method relates to processing of a silicon substrate, including a silicon-on-insulator (SOI) wafer. Repeated masking and etching steps ensures precise microfluidic layout, including a flow junction between microfluidic channel(s) and reagent channel(s), along with a sampling port for introducing analyte from the surrounding environment to the microfluidic channel(s).

In an embodiment, the invention is an implantable electrochemical sensor for sampling and/or detecting an analyte, including an analyte relevant in an in vivo biological system, such as a method of analyzing an analyte in a biological sample with any of the sensors provided herein implanted in a biological tissue. The implantable electrochemical sensor can achieve a desired total sensing time by selection of sample inflow volumetric flow-rate and periodic use of the reagent channel to ensure proper ongoing sensor operation. Relevant and reliable operation lifetimes include an in-vivo or in-situ time greater than one hour, including without having to actively manipulate the sensor, such as removing from the biological environment or changing out fluid reservoirs/solutions.

In an embodiment, the invention is a method of making any of the sensors described herein, including by processing a silicon-on-insulator (SOI) wafer so that the electrochemical sensor body has the described micro-sized or less dimensions suitable for biological implantation. In this manner, the fluidic channels can be both small, yet withstand high pressures, including on the order of up to 5 atmospheres so that fluid can be appropriately driven through the channels with appropriate flow-rates, including under laminar flow.

In an embodiment, provided herein are implantable biomedical electrochemical sensors for sensing an analyte of interest potentially found in the biological environment. The sensor can comprise an integrated on-chip probe body having a distal tip end, a proximal end and a microfluidic channel extending between the distal tip end and proximal end. The microfluidic channel is embedded within a probe body, including in at least the distal end having a cross-section configured for implantation into biological tissue. The configured for implantation refers to a sufficiently small cross-section to avoid undue tissue damage. For example, the cross-sectional area can be less than or equal to 10,000 µm², and more preferably less than or equal to 1,000 µm². In this manner, the probe is configured for long-term in vivo implantation without substantial tissue damage, including in a brain. The general shape, and components thereof, include those provided in in U.S. Pat. Pub. No. 2021/0393175 (Atty Ref. 338843: 73-20 US) filed Jun. 23, 2021, which is specifically incorporated by reference.

The electrochemical sensors are compatible with membrane dialysis applications, including by providing a membrane in fluidic contact with the microfluidic channel for membrane dialysis. The membrane may be configured to selectively pass only analyte of interest while ensuring unwanted material does not enter the microfluidic channel. This can further improve and extend electrode functional, particularly in harsh conditions such as associated with in vivo applications.

The implantable biomedical probe can be configured to detect an analyte in the sample fluid at a low concentration range, depending on the application of interest. For single or few sensor microelectrodes, detection limits can be as small as about the 1 µM to 100 µM range, including about 10 µM. By increasing the surface area of the microelectrodes, including by using multiple microelectrodes, including in one or more microfluidic channels, much lower detection ranges are possible, including as low as 10 nM and corresponding to fast scan cyclic voltammetry (FSCV). P. Puthongkham. “Recent Advances in Fast-Scan Cyclic Voltammetry.” Analyst. 2020 Feb. 17; 145(4): 1087-1102. Desired detection limits are achieved by a combination of various parameters, such as flow rates, microfluidic channel size and length, electrode material and size. For example: if the electrode is made longer (to increase its surface area), then it takes more time for a given chemical “peak” to traverse the full length of the electrode. If the sampling channel splits into several channels in parallel for electrode placement and detection, the splitting into parallel channels effectively “splits” the applied flow rate. All this being said, increasing the applied flow rates could result in restored temporal resolution when increasing electrode area.

The implantable portion of the sensor may have a distal tip geometry and the microfluidic channel a fluidic characteristic to provide a temporal resolution of 1 second or better and a spatial resolution of 100 µm or better. The tip geometry may be an orifice area, effective diameter, and/or shape. The fluidic characteristic of the microfluidic channel may be length, cross-sectional area, effective diameter, flow-rate and/or relative flow-rates between different channels, such as between an immiscible or miscible fluid channel and the fluid channel. Any of the sensors and related methods provided herein may control the relative flow-rates in different channels. For example, the flow-rate ratios between channels may relate to miscible and immiscible solutions and flow in the microfluidic channel. Flow-rate control of a channel carrying a miscible solution is useful for controlling dilution rates. Flow-rate control of immiscible solutions are useful for sample segmentation/droplet generation/2-phase flow, including as described in US Pat. App. No. 17/356,062 filed Jun. 23, 2021.

Any of the implantable sensors may have a unitary material that comprises a silicon substrate, including a silicon-on-insulator (SOI) substrate.

Also provided herein are methods of analyzing an analyte using any of the electrochemical sensors described herein and also methods of making any of the electrochemical sensors described herein.

In an embodiment, the electrochemical sensor for detecting an analyte comprises a microfluidic channel having a lumen surface extending between a proximal end and a distal end to define a microfluidic lumen. A microelectrode forms a portion of the lumen surface and is configured for fluid contact with a fluid sample that flows in the microfluidic lumen to detect the analyte, if present, in the fluid sample as the analyte flows by the microelectrodes. As desired the microelectrode surface may be functionalized to facilitate interaction with the analyte and thereby improve electrical signal output. A sampling port is fluidically connected to the microfluidic channel distal end and configured to introduce the fluid sample from a sampling area adjacent to the sampling port to the microelectrode of the microfluidic lumen. A reagent channel is fluidically connected to the microfluidic channel, wherein the reagent channel is configured to introduce a reagent solution to the microelectrode for microelectrode calibration and/or cleaning. A flow controller is fluidically connected to the microfluidic channel and/or the reagent channel to control flow of the fluid sample through the sampling port and the microfluidic channel. The microfluidic channel, sampling port and reagent channel is formed from a unitary substrate, such as a Si substrate, wherein the microfluidic lumen has an effective radius as small as 4 µm and capable of withstanding a high pressure during fluid flow, such as a pressure of up to 4 atmospheres, without leakage.

A plurality of microelectrodes may be used for multiplex detection of a plurality of analytes from the fluid sample introduced to the microfluidic channel. For example, each microelectrode may be independently functionalized to target a specific analyte of interest.

The reagent solution from the reagent microchannel may be introduced to the microfluidic channel to reduce microelectrode fouling associated with a fouling material from the environment surrounding the electrochemical sensor, wherein the reagent fluid does not exit the sampling port into the sampling area.

A characteristic dimension of the microfluidic channel cross-section may be less than a diffusion layer formed by the fluid sample undergoing laminar flow in the microfluidic channel. Laminar flow may refer to a Reynolds number in the microfluidic channel that is less than about 2400, less than 2000, or less than 1500.

The microelectrode may comprise one or more thin film electrodes having a thickness less than 1 µm and a total fluid contact surface area of between 100 µm² and 100 mm².

The microelectrode may be one or more of: a functionalized electrode comprising an analyte-specific recognition element, such as a polypeptide, a polynucleotide, an antibody, a molecular imprinted polymer (MIP), a carbon-fiber electrode; a parylene-C passivated electrode; a pyrolyzed photoresist; a gold electrode; a platinum electrode; an ion-selective electrode (e.g., Ag/AgCl); a boron-doped diamond electrode; and a titanium electrode.

The microelectrode may form part of a field-effect transistor (FET).

The electrochemical sensor may have a form factor configured for implantation into a living animal or person and the analyte is from a biological sample, and the microelectrode positioned in the microfluidic channel resists fouling, thereby increasing operational lifetime of the implanted electrochemical sensor compared to an electrochemical sensor that is not embedded in a microfluidic channel.

The electrochemical sensor is compatible with any of a range of reagent solutions, such as a reagent solution selected from the group consisting of: a calibration solution; a cleaning solution; an activating solution; and an electrode regeneration solution.

The reagent microchannel may be configured to convey a regeneration solution the microelectrode(s) for regenerating an electrode surface parameter without disturbing tissue surrounding the implanted electrochemical sensor. “Electrode surface parameter” is used broadly herein to refer to any parameter that is associated with microelectrode performance. For example, the parameter may refer to amount of surface contamination, surface roughness, degree of functionalization (e.g., concentration of functional material on surface), surface purity, thickness, and the like.

The electrochemical sensor may have a measurement run time of at least 90 seconds before introduction of the reagent solution to the microelectrode to provide a reset of the measurement run time, without disturbing tissue surrounding the implanted electrochemical sensor.

The electrochemical sensor may further comprising a membrane positioned upstream of the microfluidic channel for membrane microdialysis.

The electrochemical sensor may have an implanted cross-sectional area that does not adversely impact surrounding tissue, including less than 1 mm²; less than or equal to 1200 µm²; including a probe cross-section corresponding to about 15 µm × 75 µm.

The electrochemical sensor may have an in-line calibration mode to calibrate the electrode without disturbing an environment surrounding the electrochemical sensor.

The reagent channel may be connected to the microfluidic channel to form a microfluidic junction positioned between the sampling port and the microelectrode.

The electrochemical sensor may further comprise one or more flow controllers operably connected to the microfluidic channel proximal end; and/or a proximal end of the reagent channel. The flow controller may be a variable pressure pump to control pressure and corresponding flow-rate and flow direction in each of the microfluidic channel and reagent channel, optionally without any flow valves, wherein the variable pressure pump generates a fluid sample flow rate through the microfluidic channel that is between 1 nL/min and 300 nL/min.

The electrochemical sensors provided herein can be used to detect analyte at flow-rates less than 50 nL/min.

The flow controllers may be configured to provide a plurality of electrochemical sensor modes, the modes comprising: a sampling mode; a calibration mode; a regeneration mode; a cleaning (rinse) mode; and a transient mode.

The electrochemical sensors provided herein are useful in a range of applications, including but not limited to: an in-brain sensor; a continuous glucose sensor; an oxygen reduction sensor in a fuel cell; a water quality sensor; a toxin detector; a corrosion sensor; a scanning electrochemical microscopy probe; a pH sensor; an impedance sensor; a component of a battery; or a component of a desalination device.

An example of a preferred form factor includes having the sampling port positioned in an insertable portion having a needle geometry, and the sensor is positioned in a non-insertable potion, and the average thickness of the insertable potion is less than the average thickness of the non-insertable portion, including at least by a factor of at least 10.

The electrochemical sensors may be characterized as having a spatial resolution that is better than 10 µm (e.g., by selecting an inlet dimension and/or flow-rate) ; a temporal resolution of less than 1 second; and an operational lifetime greater than 1 hour (via the embedded microelectrodes).

The electrochemical sensor may be integrated with any of the push/pull neural probes described in US Pat. App. No. 17/356,062 (Atty ref. 338843: 73-20 US) filed Jun. 23, 2021, which is specifically incorporated by reference herein for the devices and methods disclosed therein.

Also provided herein is a method of using any of the disclosed electrochemical sensors, the method comprising one or more of: chemical separation (e.g., capillary electrophoresis); coupling the electrochemical sensor to external machinery (e.g. localized electrical contact for electrospray/electrodeposition for coupling to mass-spectrometry); non-contact sensing (e.g., capacitance sensing); fluidic manipulation (e.g. electro-osmosis for direct-contact, dielectrophoresis for non-contact); and/or interactions between electrochemical cells (e.g., positive feedback for increased sensitivity). This range of method use reflects the sensors provided herein have a range of applications.

Also provided are methods of making a monolithic in-line electrochemical sensor, such as by: patterning a layer of SiN on a Si substrate, wherein pattern openings in the SiN layer correspond to microelectrode electrode positions; depositing a metal layer over the patterned layer of SiN and exposed Si substrate corresponding to the microelectrode positions; forming a microfluidic network of channels in the Si substrate by buried channel definition using passivation, etch-hole definition and etchant introduction; and plasma etching to form contact pads in electrical contact with the metal layer electrodes adjacent to a portion of the channel; deep Si etching to define an overall geometrical shape of the in-line electrochemical sensor, including a square base with a needle to receive a fluid sample; providing fluid connectors to the microfluidic network of channels for fluid flow control in the channels.

The microfluidic network of channels may comprise a microfluidic channel with the microelectrodes having a cross-sectional shape that is semicircular with a radius of less than or equal to 20 µm.

Also provided are methods of detecting an analyte by inserting any of the electrochemical sensors described herein into a subject at an implantation site and introducing a biological sample from the subject to the microfluidic channel via the sampling port. The microelectrodes are energized and an electrical output from the microelectrode detected so that the presence or absence of the analyte is detected based on the electrical output from the microelectrode. In this manner, the microelectrodes may be configured as part of a FET.

The method may further comprise the step of: controlling a relative pressure between the reagent channel and the microelectrode channel to flow the reagent fluid through the microelectrode channel; wherein the reagent fluid is a cleaning solution or a calibration solution, thereby increasing an operational lifetime of the implanted electrochemical sensor to 1 hour or greater without having to remove the electrochemical sensor from the implantation site.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A is a schematic illustration of one embodiment using the proposed technology: an integrated sampling probe with in-channel electrochemical sensors and microfluidics for on-chip detection of neurotransmitters and in-situ electrode calibration. FIG. 1B is a cross-section of the monolithically fabricated in-channel electrodes for sensing neurochemicals in from sampling area in flow. FIG. 1C is a close-up view of the reagent channel fluidically connected to the microfluidic channel at a microfluidic junction and corresponding components. FIG. 1D illustrates an overall form factor, including with the sampling region (e.g., the inlet port and associated channel) that is physically separated (e.g., decoupled) from the sensor region.

FIGS. 2A-2D. Monolithically integrated in-channel electrodes. FIG. 2A is a fabrication schematic (not to scale, pink = SiN, yellow = Ti/Pt, gray = Si). FIG. 2B is a SEM image of a channel cross-section with 5 µm radius (top) and exposed Ti/Pt on the underside of the channel cover (bottom). FIG. 2C is a photo of multiple channels in parallel, interdigitated WE and RE/CE electrodes, and contact pads. FIG. 2D is an assembled and packaged device with electrical and fluidic interfaces.

FIG. 3A. Cross-section of microfluidic channels; FIG. 3B is a cross-section of a microfluidic channel with a droplet generator and FIG. 3C is a cross-section of microfluidic channel with sampling needle with cross-section of 15 µm × 50 µm.

FIG. 4. Schematic of flow redirection for realization of different modes enabling detection of samples (sampling), pump/solution switching (transient), and addressing electrodes non-invasively (calib/regen/rinse).

FIG. 5 Hydraulic circuit model (top panel) of valve-less flow redirection system (bottom panel).

FIG. 6A contains CVs at different flow rates at 100 V/s. FIG. 6B shows the linear relationship between peak current and scan rate observed for all flow rates tested. FIG. 6C is the non-monotonic peak current enhancement with flow rate demonstrated at multiple scan rates.

FIGS. 7A-7C. Fabrication recipe for the in-channel electrochemical sensor, FIG. 7A being the main pipeline to yield a silicon sampling probe, and FIG. 7B being the steps to yield in-channel electrodes, compatible with FIG. 7A. FIG. 7C optical (top) and SEM cross-section (bottom) of in-channel electrodes.

FIG. 8. Schematic illustrations of the sensor, channel and related fluid-flow, for different applications relevant for the electrochemical sensors provided. Examples include: electrochemical sensing, chemical separation, coupling to external components for additional analysis/characterization, non-contact sensing, fluidic manipulation and electrochemical feedback by interactions between electrochemical cells.

FIG. 9A Current-Voltage (CV) plots at different scan-rates with inset showing linear fit for peak currents. FIG. 9B CV peak current vs. concentration with inset showing linear calibration curve.

### DETAILED DESCRIPTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In general, the terms and phrases used herein have their art-recognized meaning, which can be found by reference to standard texts, journal references and contexts known to those skilled in the art. The following definitions are provided to clarify their specific use in the context of the invention.

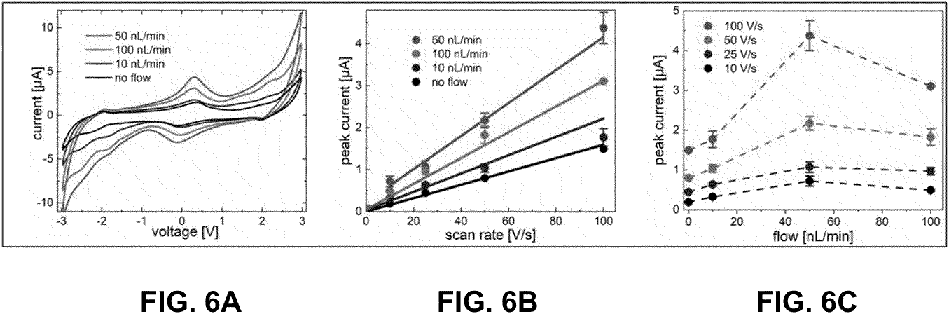

“Microfluidic channel” refers to a conduit having walls that define a lumen in which liquid fluid can flow without unwanted leakage. The lumen has a cross-sectional area with an effective radius that is less than about 100 µm, including less than 50 µm, less than 30 µm, less than 10 µm, and preferably, between about 3 µm and 10 µm in radius. In one aspect, as described herein, the diameter of the microfluidic channel is preferably less than the diffusion layer in the channel for laminar flow (e.g., Reynolds number less than 2000, preferably less than 1000), so that analyte has the opportunity to interact with the electrode surface simply on the basis of diffusion because the maximum separation distance from the electrode surface is such that the analyte can diffuse to the electrode surface before the analyte flows past the microelectrode. In this aspect, the sensor may be described as having a “thin-layer cell”. See, e.g., FIG. 6B as validation of the “thin-layer regime.”

The microfluidic channel also contains the microelectrode. “Microelectrode” refers to an electrode that is positioned on a lumen-facing surface of the microchannel. The surface area can be micron-sized, such as about 100 µm² to 1000 µm². Of course, much higher surface areas can be achieved by using a plurality of microfluidic channels, each having at least one microelectrode positioned therein. The channels can be arranged in parallel, in series, or a combination of parallel and series. In this way, an effective surface of the microelectrodes can together readily surpass 1 mm², including up to 100 mm².

“Fluidically connected” refers to two components connected in such a manner that a fluid can flow between the components without adversely impacting the functionality of each component, such as a fluid sample, a reagent solution, and the like. Preferably, the fluid comprises a liquid that conveys one or more analytes of interest.

The term “implantable” refers to the sensor that is configured to cause minimal or no observable damage during or after implantation into a biological tissue. The term recognizes that any act of implantation will cause minor irritation or disruption to tissue, but such disruption is not necessarily observable to the naked eye. Furthermore, there is no significant and prolonged immune response, clotting activity, or the like. In other words, the probe can be implanted for a time period and then removed, without lasting or permanent damage to the subject. This is in contrast to conventional devices, where the size of the system required to accommodate all the components provided herein can result in observable adverse events, at least in part due to the size of the device. Alternatively, even for smaller sized conventional devices, the device may rapidly degrade in performance, requiring periodic calibration (to accommodate drift) and/or cleaning, which can in itself disturb surrounding tissue. Such disturbances may include blunt force trauma and associated tissue damage, including blood vessel damage, immune response, scarring and up to an including observable tissue damage and death. Such damage can be quantified by measuring tissue damage markers, including proteins, enzymes and immune cells, depending on the biological tissue type. The damage/no damage may be quantifiably defined, such as a difference of at least 5%, 10% or 20% of one or more biomarkers in the implantation region compared to the value before the implantation. The electrochemical sensors provided herein provide the functional benefit of having small cross-sectional areas of insertion while still being “integrated” in that each of the cleaning and calibration functionality is maintained within the body formed of one material, preferably silicon.

“Integrated on-chip” refers to the sensor body that is formed from a common starting substrate, such as a silicon or SOI wafer. The fluidic components for droplet generation and biological sampling are formed by processing the SOI wafer, including by repeated use of photoresist layer(s) (PR), patterning and etching. See, e.g., U.S. Pat. App. No. 17/356,062 (Atty ref. 338843: 73-20 US) filed Jun. 23, 2021. The term “unitary material” is used herein to refer to a single material that forms the common starting substrate, such as silicon, doped silicon, including SOI wafer.

“Probe body cross-section” refers to the cross-section of the probe that is configured for insertion into biological tissue. Depending on the application of interest, it preferably has a very small area, such as less than 10,000 µm², less than 1,000 µm² and even less than 500 µm² or 100 µm². The relatively high strength silicon provides the necessary strength to ensure the probe does not break during use. Depending on the tissue type, the cross-sectional area for insertion may be relaxed. For example, for brain insertion, the cross-sectional area may be on the small side to minimize brain injury; for skin insertion, the cross-sectional area may be larger as the risk of adverse impact due to skin injury is less than for brain. Accordingly, a portion of the probe body (e.g., the proximal end) may be relatively large because that portion need not be implanted, with another portion (e.g., the distal end or distal tip end) may be relatively small as that portion is implanted.

“Biological fluid” is used broadly herein to refer to fluid that is collected by the probe during implantation. The fluid can be intracellular fluid, extracellular fluid, synovial fluid, sweat, blood, saliva, tears, urine, and any other fluid associated with a biological tissue. “Analyte” and “chemical” are intended to be used interchangeably and refer to a substance within the biological fluid that can be detected by the buried electrode of the electrochemical sensor. For example, in brain tissue the analyte may be a neurotransmitter and/or metabolites thereof.

“Reagent solution” refers to a fluid that can be introduced to the microfluidic channel to improve or maintain an electrode parameter, such as calibration status, sensitivity, accuracy, and surface quality. Accordingly, “calibration solution” refers to a solution having a known quantity of a material that can be used to calibrate the microelectrode. In this manner, electrode drift can be readily accommodated for as the output of the electrode is tied to a known quantity. “Cleaning solution” refers to a solution that can remove contaminating material from the electrode surface. “Activating solution” refers to introduction of a material in the reagent solution to the analyte to ensure the analyte is detectable by the electrode. For example, mixing a derivatization molecule (introduced via reagent channel) with a non-electroactive sampled analyte can be used to make the sampled analyte of interest electroactive (i.e. able to be detected). One example pair is Amphetamine detection (analyte) with 1,2-naphthoquinone-4-sufolnate (derivatization). See, e.g., Parrilla et al. “Derivatization of amphetamine to allow its electrochemical detection in illicit drug seizures.” Sensors and Actuators B: Chemical. 337, 15 Jun. 2021, 129819. “Electrode regeneration solution” refers to a solution that is configured to restore the electrode surface. This is relevant for situations where the electrode is degrading and losing material. The can be addressed herein by providing an electrode regeneration solution with an electroplating method to deposit metal onto electrode surface by applying potential to medium with ions of the corresponding metal provided by the electrode regeneration solution.

“Electrochemical sensor modes” refers to the ability of the instant sensors to accommodate different modes so as to achieve a prolonged operational lifetime. As discussed, conventional electrochemical sensors often have limited operational lifetimes and/or require frequent cleaning/calibrations, making them ill-suited for prolonged in-vivo use. A “transient mode” of the instant sensor essentially de-pressurizes the reagent channel, allowing for external fluidic connections to be dis/reconnected without disturbing the downstream fluidic circuit. With this mode, reagent chemistry is readily changed (e.g. different concentration for calibration, rinsing medium, regeneration medium, derivitization medium, etc.) without disturbing the rest of the sensor or the environment in which the sensor is implanted.

“Heavily-doped” refers to a semiconductor, such as silicon, having 10¹⁷ cm⁻³ or greater, including an impurity concentration of about 10¹⁷ cm⁻³ to 10²⁰ cm⁻³, and any subranges thereof.

The invention can be further understood by the following non-limiting examples.

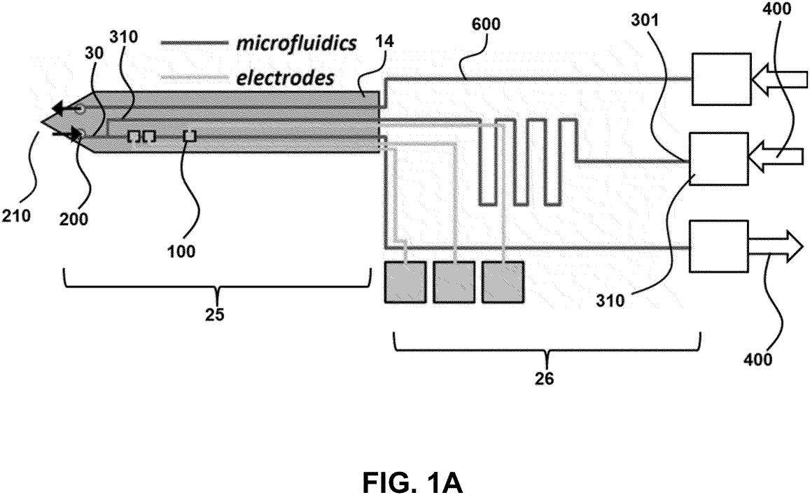

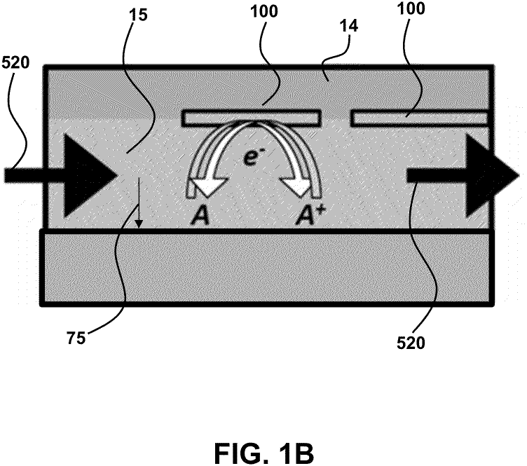

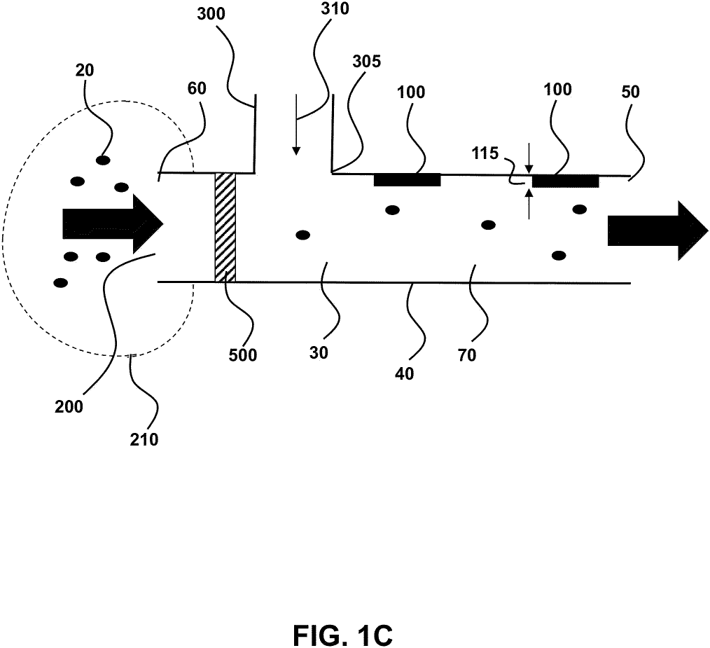

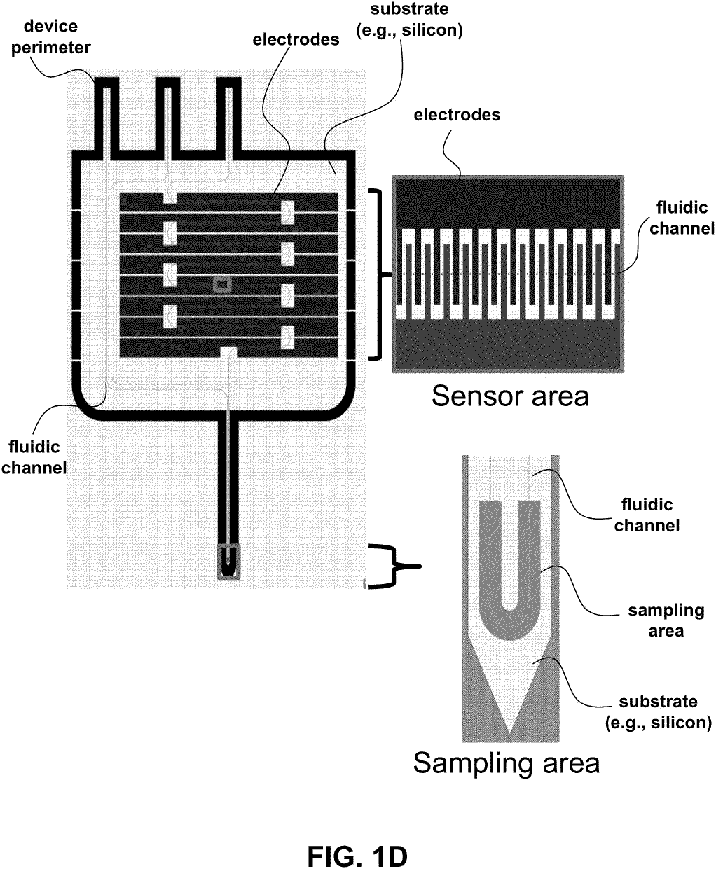

The various figures provide one illustrated embodiment of the device, including FIGS. 1A-1D that illustrates an electrochemical sensor formed of a unitary substrate 14, such as silicon (Si), that provides a microfluidic channel 30 to which a fluid sample 15 that may contain an analyte 20 is introduced. The microfluidic channel 30 has a lumen surface 40 extending between proximal 50 and distal 60 ends to form a microfluidic lumen 70. One or more microelectrodes 100 form a portion of the lumen surface 40 for electrochemical detection of analyte 20. The microelectrodes may be a thin film electrode having a thickness 115. A sampling port 200 if fluidic contact with microfluidic channel distal end 60 is configured to facilitate passage of analyte 20 from a sampling area 210 to the lumen 70 and attendant microelectrodes 100. The sampling area is also described herein as being “adjacent” to the sampling port 200. The precise area depends on the application of interest, but functionally is defined as that area wherein an analyte is capable of entering the microfluidic region over the relevant time period. A reagent channel 300 is configured to introduce a reagent solution 310 to the lumen 70 of microfluidic channel 30. The microfluidic channel may be described as having an effective radius 75. FIG. 1C is a close-up view of the microfluidic junction 305 formed between the reagent and microfluidic channels (30 300). Flow controllers 400 (schematically illustrated with arrows) may connect to the microfluidic channel and/or reagent channel at or toward proximal ends (50 301). Additional inlet channel 600 may be provided to introduce material to the biological tissue and near the sampling port and/or to help control or drive fluid flow through channel 30 from sampling area 210. The electrochemical sensor may have a form factor 25, such as needle geometry, to facilitate insertion into biological tissue while minimizing unwanted tissue damage. As illustrated in FIG. 1A, there may be a tapered tip at the distal end of the microfluidic channel, extending a longitudinal distance toward the body of sensor 26, with both the form factor portion 25 and rest of body 26 formed of unitary material 14, such as Si. A preferred from factor is one where the characteristic dimension of the inserted portion, such as width, is less than the longitudinal distance corresponding to length of portion 25 in FIG. 1A, such as at least by a factor of 2, 5, or 10. As desired, a membrane 500 may be used.

With respect to the form factor of the electrochemical sensor, the ability to have different substrate thicknesses for different components of the electrochemical sensor is advantageous, particularly for biological sampling. For example, portion 25 corresponding to the implanted portion, may have a thickness less than the remaining non-inserted portion 26. The thinner portion minimizes tissue damage whereas the thicker portion improves robustness of the overall sensor, to facilitate handling of the sensor without damage. The inserted portion 25 may have a thickness less than 50 µm, and the non-inserted portion 26 may have a thickness greater than 300 µm. Accordingly, form factor includes a thickness of the implantable portion 25 that is less thick than that of the portion 26 that is not inserted, thereby facilitating minimal damage to the material in which the sensor is implanted while also increasing resistance to sensor damage during handling/manipulation, such as movement of sensor to insert the insertable portion into the material, including a tissue material For example, a thinner thickness of about 15 µm and a thicker thickness of about 400 µm - 500 µm. In an embodiment, the substrate comprises Si, including a doped or an undoped Si substrate.

Another aspect of form factor relates to the sensor portion of the electrochemical sensor that is able to be positioned near the sampling port 200. In this manner, dead volume between sampling and detection portions is minimized. In other embodiments, the electrochemical sensor can be placed on the proximal portion of the substrate, including that portion labelled as portion 26, where there are essentially no limits on physical placement. This allows for some embodiments where the area of the sampling port 200 can be made very small (high spatial resolution), while the electrode sensor area 100 can be made very large (high sensitivity). See, e.g., FIG. 1D. This decoupling of sampling and sensor areas allows for high design flexibility when manufacturing devices with specific performance needs.

### Example 1: Electrochemical Biosensor

This example describes a chemical sensor made to address the issues of long-term performance in conventional in-vivo electrochemical methods by leveraging microfluidics and semiconductor manufacturing processes. The sensor’s electrodes are resistant to fouling and have the ability to be calibrated and regenerated in-place without disturbing the sample of interest. The exemplary application involves in-brain sensing. As described, however the advantages of this technology does, of course, apply to a range of electrochemical sensing applications. Examples include, but are not limited to, continuous glucose sensing, oxygen reduction in fuel cells, limestone erosion near groundwater sources, and toxin detection in water purification.

Neurochemical sensing for treatment of disease and disorders: Detection of electrical and neurochemical transients in live and awake animals leads to better understanding of how underlying brain circuits function^{1,2} and affect our daily behaviors³, while also contributing to the development of several drugs and treatments that alleviate chemical imbalances present in many neurological disorders and disease^{4–6}. This is because the fundamental signaling modality of the central nervous system involves chemical exchange amongst its constituent cells, whether it be ionic currents across neuronal cell membranes (sensed via electrophysiology), synaptic transmission, or extrasynaptic release/uptake of neurotransmitters, neuropeptides, and neurohormones^{1,2}. Increasing spatiotemporal resolution of current sensors has been a massive and continual effort in literature, as localized detection (<100 µm × 100 µm × 100 µm) at time scales fast enough to capture quick (<1 s) neurochemical volume transmission dynamics provides higher quality information which can further elucidate neural circuit function and targets for treatment development.

Technologies for fast and sensitive detection in-vivo: Electrochemical methods such as fast-scan cyclic voltammetry (FSCV) and chronoamperometry are the more popular approaches regarding in-situ neurochemical detection, as they have achieved in-vivo limits-of-detection as low as ~10-100 nM with probe dimensions on the order of 10 µm with ~100 ms temporal resolution for electroactive chemicals⁷, most notably dopamine (DA) and serotonin (5-HT)⁸. Compared to off-site measurement methods however, electrochemical methods traditionally can only target electroactive molecules, and have limited selectivity and multiplexing capabilities. Functionalizing electrodes with molecule-specific enzymes has allowed for non-electroactive species such as glucose, glutamate, lactate, and acetylcholine to be measured thru electroactive by-products with high selectivity⁷, although typically the temporal resolution worsens to around ~1 s due to series addition of slower enzyme reaction kinetics^{9,10}. Other molecule-specific recognition elements like DNA aptamers or molecular imprinted polymers (MIPs) have shown to facilitate selective transport of analytes close to the electrode/sensing surface, decreasing signatures from interfering species^{11–13} and enabling chemical sensing field-effect transistors (FETs) to overcome the once debilitating screening effect of the electrical-double layer^{14–16}. However biomolecules such as enzymes and DNA aptamers show instability over long periods of time in vivo, and MIPs face unanswered questions in biocompatibility and toxicity¹³, leaving FETs largely out of current in-vivo sensing systems. Therefore, in-vivo electrochemistry presents the most promising of neurochemical detection methods for translation to long-term studies, however it carries several disadvantages. One being fouling and degradation of the electrode over time, resulting in short measurement windows due to drift in electrode sensitivity. This is coupled with the inability to calibrate or regenerate electrodes in-situ, leaving most methods (namely, FSCV) restricted to transient (rather than basal) concentration measurements.

Fouling degrades electrochemical sensor performance: Fouling of the electrode surface owes to a biological media filled with corrosive and adsorptive molecular species exacerbated by the foreign body response, as well as the adsorption of electrochemical reaction byproducts. Upon insertion of the probe, the brain suffers an acute and chronic immune response, where microglia, astrocytes, and macrophages (if the blood-brain barrier is broken) attach to the probe and secrete degradative enzymes and reactive oxidant species in attempts to isolate and break down the foreign body^{5,17–} ²³. Sensing electrodes are typically fabricated on the distal tip of implanted electrochemical probes in direct contact to this harsh environment of damaged tissue, hence fouling is seen to accelerate in-vivo. Minimizing tissue damage by decreasing the cross-section of the probe as well as choosing more biocompatible, soft, and robust electrode and probe passivation materials (e.g. carbon fiber electrodes and parylene-C passivation) have shown to mitigate the immune response and the related decrease in electrode performance over time^{24,25}. While the current state of the art carbon-fiber electrode lifetime of 100 days represents a huge hope for chronic applications, fouling/degradation still led to a decrease in sensitivity, electrochemical drift, and meaningful experimental time windows of 90 seconds or less²⁶.

Electrochemical drift hinders accurate measurement: Electrochemical drift describes the changes in both faradaic and non-faradaic current-voltage responses that occur over tens of seconds due to adsorption of foulant species, etching of the electrode surface, changes in electrode surface functional groups, and ionic fluctuations within the solution to be sensed (e.g. pH changes)^{8,26–30}. The faradaic currents are of interest, as they are used to quantify neurotransmitter concentration. However, due to the large (>100 V/s) scan-rates in FSCV, the non-faradaic/capacitive currents are typically thousands of times higher than faradaic currents generated from electrolyzed neurotransmitters at basal concentrations. Witnessing that within short (<90 s) experimental time-windows non-faradaic currents are relatively stable, subtraction of the background signal was adopted as a standard method to mitigate the non-faradaic contribution³¹. In-vivo background signals include basal concentrations of neurotransmitters, which limits background-subtracted FSCV to measurements of concentration transients. The large current background still drifts slowly over time, and because of this FSCV requires a new background signature every 90 seconds²⁶, leaving slower transients of neurochemicals indistinguishable. Recently, statistical techniques which attempt to predict and subtract drifting background signals look especially promising^{8,26,32,33}, however training data is gathered in-vitro rather than in-vivo, and typically leaves out drift from the faradaic component. Because of drift, frequent calibration is required for accurate measurements over time, however calibration of in-vivo electrodes is impossible in-situ, and is done in-vitro before and after implantation, which raises questions about accuracy of long-term measurements.

The above problems are addressed herein by use of on-chip integrated microfluidic electrodes, which provides a number of functional benefits.

On-chip integrated microfluidic electrodes for sensing chemicals in-situ: Sampling from the brain (or any other medium of interest) with off-site detection can alleviate many issues of in-situ electrochemical sensors, as the bulk of the immune response (i.e. harsh environmental conditions) is left to the sampling area as opposed to the sensing area. Microfluidic push-pull and microdialysis probes have served as bulk and selective chemical samplers respectively, and sampled fluids from the brain have been successfully transferred off-probe to pre-treating, separation, sensing, and multiplexed analysis systems such as electrospray ionization-coupled mass spectrometry³⁴, liquid chromatography coupled mass spectrometry^{34–36}, inductively coupled plasma mass spectrometry³⁷, and liquid chromatography-coupled electrochemistry³⁸. Large footprint (~500 µm diameter) and slow temporal resolution (-minutes) compared to their electrochemical counterparts have traditionally held them back until recent endeavors in silicon microfabricated neural probes have integrated complex microfluidics on-chip for several advantages, such as segmented flow post-sampling to improve temporal resolution to <10 seconds while miniaturizing sampling probe dimensions to ~100 µm diameter to minimize invasiveness and the inflammatory immune response^{7,34,36,39–43}.

This example demonstrates a microfabricated silicon total analysis system with integrated microfluidic sample collection/flow-redirection and in-channel electrochemical sensing, with several key innovations, including:

1: Reduced electrode fouling by fluid flow and separation from harsh environments: In-channel electrodes are: (i) physically separated from the sampling site (e.g. inflammatory tissue response in the example use case); (ii) immersed in a flowing solution which further reduces fouling; and (iii) have access to electrochemical methods that take advantage of convective flow.

2: Valve-less on-chip flow switching for electrode calibration and regeneration in-situ: Through microfluidic design and flow redirection, calibration and cleaning of in-channel electrodes are addressed by flowing “reagent solutions” to the on-chip cell.

3: Monolithic integration of microfluidics and in-channel sensors that is highly scalable: Through leveraging advanced silicon microfabrication techniques, we introduce a novel monolithic in-channel electrode protocol which adds minimal thickness (~100 s of nm, thickness of the thin-film electrodes) to the overall fluidic sensor. This means that the in-channel electrodes are readily integrated with ultra-thin sampling needles for minimum tissue damage and complex microfluidic channel networks for passive flow-switching. Other technologies typically use substrate-to-substrate bonding to yield in-channel fluidic sensors, yielding total thicknesses that are 100’s-1000’s of µm in thickness, which must be thinned down with additional processes, such as chemical-mechanical polishing (CMP), if they are to be used for in-tissue sensing.

4: Sensitivity enhancement using flow in a thin-layer cell configuration: We demonstrate a novel enhancement of peak currents in FSCV measurements by utilizing flow in an embodiment of the device which has a “thin-layer” configuration, which occurs when the channel height is smaller than the diffusion layer above the electrode within the given experimental time frame (see Example 5 for details).

These innovations provide an on-line addressable electrochemical probe capable of longer experiment times, in-situ electrode calibration and regeneration, and basal concentration measurement with FSCV. Samples collected with this system are compatible still with off-chip analysis, as is with traditional sampling probes. Further still this technology is compatible with electrode surface functionalization techniques to target specific chemicals/use cases. Finally, the in-channel electrodes can also be readily used in other applications, which require liquid-to-electrode contact, such as electrophoresis, water electrolysis, electrospray ionization, electrodeposition of liquid droplets, and the like.

### Example 2: Monolithic Fabrication of Integrated Microfluidic Electrochemical Sensor

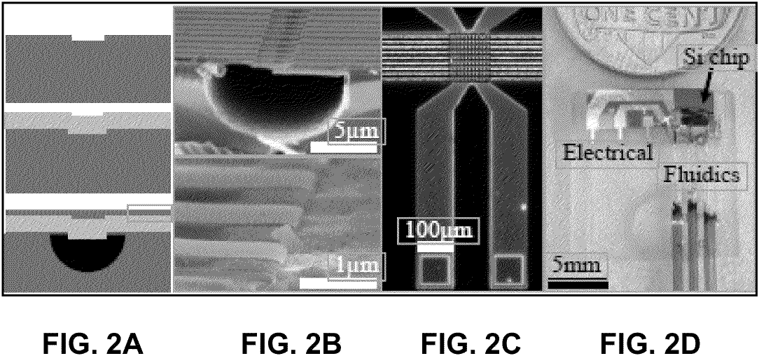

To sense channel contents via electrochemistry, the electrodes must make direct contact with electrolyte solutions flowing within the microfluidic channels. A typical method to define electrodes within silicon channels involves wafer-to-wafer bonding, but that process is limited in terms of feature alignment and thus far limited to a thickness of just over 100 µm⁴³. Provided herein is a novel fabrication method which yields in-channel electrodes in a monolithic manner, improving issues of alignment and scalability without adding any substrate thickness. Briefly, the process involves defining where electrodes would be exposed to the microchannel contents (FIG. 2A, top panel), electrode definition + metal deposition (FIG. 2A, middle panel), and buried channel definition using passivation, etch-hole definition, and XeF₂ etching (FIG. 2A, bottom panel). Contact pads are then defined, and deep silicon etching using the Bosch Process is used to define the probing chip’s shape and plumbing connections. The passivating SiN layer between the Si substrate and electrodes is typically about 100 nm in thickness. After the electrode patterning and channel definition, the channels are “sealed” with an additional 2-4 µm SiN layer.

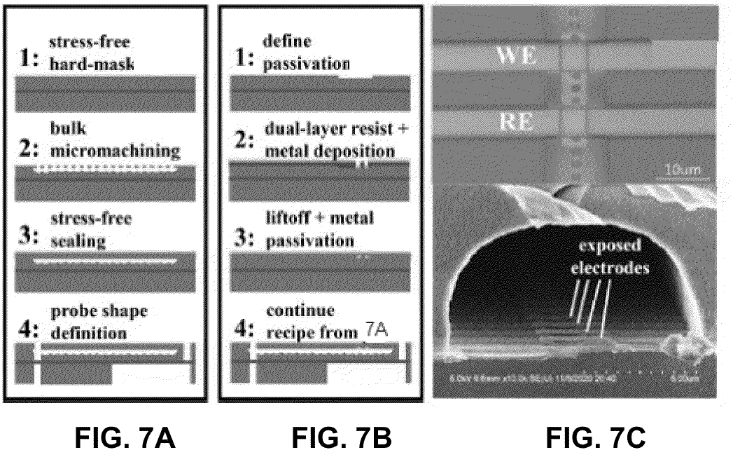

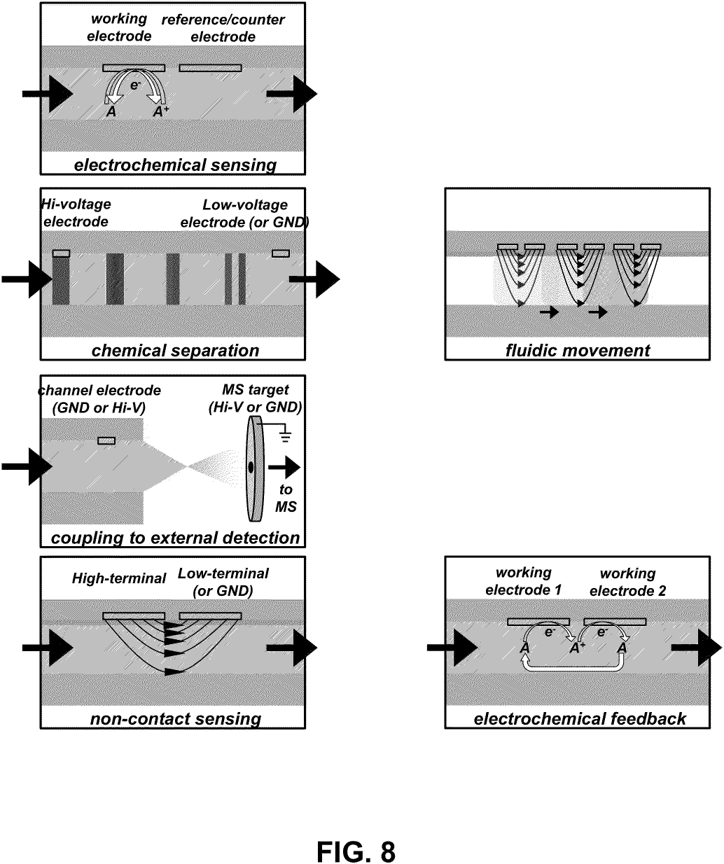

FIGS. 7A-7C is another schematic summary of a method of making that provides electrodes within silicon needle substrates of just 15 µm thickness in a monolithic manner, improving issues of alignment and scalability. Briefly, the process involves defining where electrodes would be exposed to the microchannel contents (FIG. 7B1), electrode definition + metal deposition (FIG. 7B2), liftoff + metal passivation (FIG. 7A3) and the subsequent XeF2 etching, sealing and deep-reactive ion etching (DRIE) steps from our silicon sampling probe recipe (FIG. 7B). The electrodes were shown to be exposed to channel contents by measuring changes in solution resistance upon pumping electrolyte solutions of several conductivities (preliminary), as well as SEM imaging of the cross-section (FIG. 7C). The devices can be used for electrochemical sensing and characterization experiments.

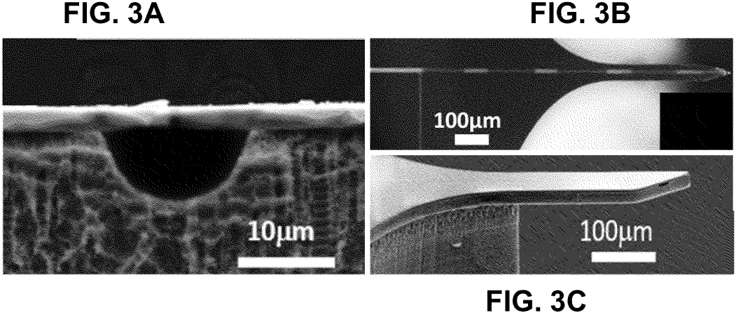

In some embodiments, the technology may be directly integrated with a minimally-invasive sampling device to decrease tissue damage and increase spatial resolution, as well as complex microfluidics to achieve complex outcomes (such as valve-less flow redirection). One example relates to a silicon-based push-pull neural probe, including as described in U.S. Pat. Application No. 17/356,062 titled “Implantable Probes and Methods of Fabrications” filed Jun. 23, 2021 (Atty Ref. 338843: 73-20 US), which is specifically incorporated by reference herein. Distinct features include embedded microfluidic channels of semicircular shape with radii of 5 µm or between 4 µm and 6 µm (FIGS. 3A-3B) and a minimally invasive sampling needle (FIG. 3C). These represent the absolute state of the art for sampling probes, being ~100× smaller than traditional microdialysis/push-pull probes. Briefly, the recipe includes stress-balanced silicon nitride deposition on a silicon-on-insulator for a mechanically strong channel cover, XeF₂ bulk micromachining for buried channels in silicon, and two-sided Bosch process for definition of the probe and needle shape. This method and the monolithic fabrication of in-channel electrodes mentioned previously are compatible by design, enabling embodiments such as that denoted in FIG. 1 (Schematic 1).

### Example 3: Valve-Less Flow Redirection for In-Situ Calibration and Regeneration of Electrodes

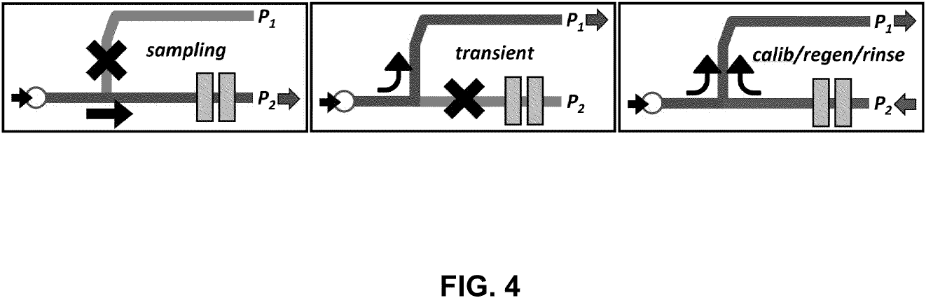

Provided herein is a passive solution for flow redirection to realize chemical calibration and regeneration in-situ, which is readily integrated into any of the microfluidic in-channel electrode technology. In-vivo electrochemical sensors suffer from drift, fouling, an inability to calibrate in-situ and an inability to measure basal concentrations with FSCV. A well-designed microfluidic channel network can be used to redirect flow in a manner that introduces calibrant and/or cleaning solutions to the in-channel electrodes without leaking out of the sampling area (FIG. 4). Microfluidics with soft materials such as PDMS utilize actuatable valves of elastic material. While that is appropriate for larger-sized systems having larger lumens, the small cross-section constraint of in-vivo probes required to minimize tissue damage dramatically raises hydrodynamic resistance. This is quantitatively illustrated by the laminar flow equations, including flow-rate in a lumen is proportional to the pressure drop and inversely related to hydrodynamic resistance (e.g., Q = ΔP/R), and under Poiseuille’s law ΔP varies with the inverse of the radius to the fourth power. Accordingly, an increase in the hydrodynamic resistance associated with maintaining flow through micro-sized vessels requires high-pressures, which are ill-suited for soft-materials (e.g., PDMS) of similar thickness. Such soft-materials are prone to deformation, detachment from the substrate, unstable flow rates and/or channel failure. Silicon’s high rigidity and high failure yield, in contrast, allows for precise flow control at high pressures. A challenge, however, is integration of active valves while keeping the cross-section small.

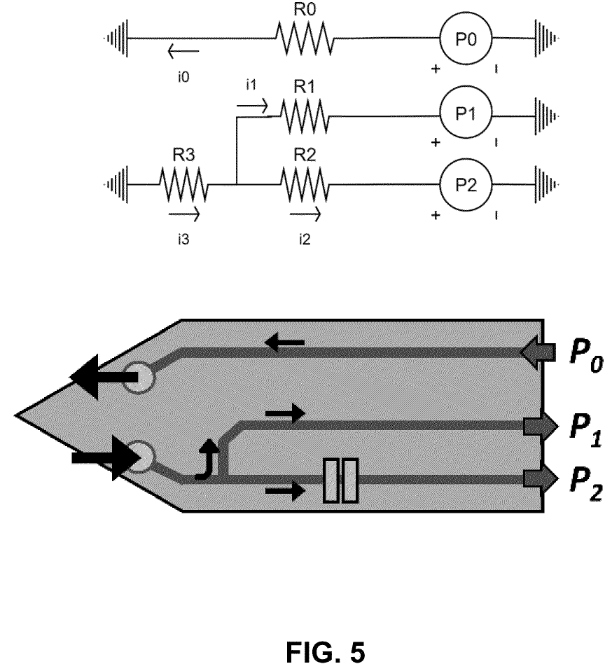

The passive solutions provided herein are validated by modeling the microfluidic channels of the sampling neural probe as an electrical circuit (FIG. 5), using the well-known analogous relationship between Ohm’s Law for circuit analysis and Hagen-Poiseuille’s Law (ΔP = 8 µLQ/(πR⁴) for laminar flow analysis⁴⁴. To achieve sampling and in-situ flow redirection, we add a fluidic bypass channel to the post-sampling channel of our push-pull neural probe (FIG. 1). In operation of a microfluidic neurochemical sampling probe, several modes are required using the same channel layout and geometry, meaning channel resistances are fixed for a given fluid once created, and pressures are varied to achieve different modes. In addition, fluids with different viscosities can be introduced to alter hydrodynamic resistances for unchanged channel layout/geometry. During the design stage, channel resistances can be widely varied alongside pressure terms to explore different situations. Outlined are five modes of operation (Table 1) for valve-less in-situ calibration/regeneration, and use variable pressure pumps and pre-determined hydrodynamic resistances to achieve all modes with one device. As a proof-of-concept, we provide a representative pumping cycle for a successfully fabricated device, as shown in Table 1. A global constraint of keeping the flow rate of the sampling area a constant 10 nL/min away from the sampling area is instilled. This is to match the “pushing” flow rate (P₀) and to ensure no leakage into the sampling area (e.g. brain tissue).

Cycles of in-situ calibration and regeneration are tested. Programmable pressure curves are used to ensure the flow rate of the sampling channel is held at a constant 10 nL/min. The sampling reservoir is held at 100 µM DA in aCSF. Of course, during use the sampling reservoir corresponds to the fluid (or tissue) from which the device samples, including via the sampling port. The pumping cycle for both processes is as follows:

Starting in sampling mode, and the concentration is detected at in-channel electrodes with FSCV with tDAwave (e.g., a wave form relevant for dopamine detection) for trials of 90 seconds until the peak current deviates from its original value by 50%. Next the pump configuration is changed to the transient mode, de-pressurizing P1. Then, depending on the process, the P1 reservoir fluid for addressing the electrodes is replaced. If calibration, aCSF with known concentration of DA (repeated for blank, 10 µM, 50 µM, 100 µM, 250 µM, 500 µM) is introduced. If regeneration: the cleaning solution to be tested (aCSF, 0.1 M KCI, or 0.5 M H₂SO₄) is introduced. Of course, the specific composition of a calibration solution is application-dependent. The sensors and methods provided herein are compatible with a wide range of calibration solutions, so long as they can be introduced to the microfluidic channel by the reagent channel.

Next, P1 is reconnected to system, and the pumps changed to the calibration/regeneration flow mode. Depending on the process, different electrochemical processes are performed. If calibration, the concentration is detected at the in-channel electrodes with FSCV. If regeneration, a 3-step stripping waveform adapted from literature can be performed, as it has shown to restore >99% of original electrode sensitivity. Manica et al. “Characterization of Electrode Fouling and Surface Regeneration for a Platinum Electrode on an Electrophoresis Microchip.” Anal. Chem. 26 Jul. 2003, 75, 17, 4572-4577. Of course, and as described for the calibration solution, the sensors and related methods are compatible with a wide range of solutions, including cleaning solutions, regeneration solutions, and waveforms used to clean and regenerate the microelectrodes and channels.

Finally, the electrodes are rinsed, and the results of the electrode addressing processes (either calibration or regeneration) are tested. This can be done by changing the pump configurations to the transient mode, and switching the P1 reservoir to a rinsing solution (in this example aCSF). Afterwards, the pumps are configured to rinsing mode, and the cells are rinsed for 1 minute. After rinsing, the pumps are returned to sampling mode and detection can be carried out as designed. For this example, the concentration of the sample reservoir is remeasured at the in-channel electrodes using FSCV with tDAwave for 10 seconds. The sample reservoir is known to be 100 µM, and thus the calibration curve accuracy is determined as the percent error between the faradaic current predicted from the fresh calibration curve, and the actual measured current. For regeneration, the faradaic current is compared to the levels prior to the initial fouling process. Both processes are tested once a day for 30 days for long-term performance investigation. In this manner, valve-less flow redirection is useful for carrying out and switching between various modes. This is an important aspect as it allows for long-term operational lifetime without removing the sensor from the implantation site, or otherwise disturbing the implantation site, to maintain good, reliable sensor operation.

### Example 4: Enhancement of Electrochemical Peak-Current Sensitivity Using Flow in a Thin-Layer Cell

We have experimentally demonstrated an enhancement in FSCV peak currents occurs when fluid flow is introduced in embodiments that reside in the “thin-layer” electrolysis regime (occurs when the channel height is smaller than the diffusion layer above the electrode within the given experimental time frame). These embodiments were confirmed to operate in a thin-layer regime by linear dependence of oxidative peak current on scan rate (FIGS. 6A- 6B). Introduction of in-channel liquid flow resulted in up to a 4× increase in sensitivity likely due to an efficient supply of analytes to the electrode vicinity, and this enhancement worsened with the increase of flow rate (FIG. 6C). We hypothesized that this occurs because at higher flow rates, the replenishing analyte molecules begin to be swept away from the electrode vicinity faster than they can undergo electrolysis. Provided specifically herein are embodiments that specifically utilize this phenomenon.

### Example 5: Efficient Electrochemical Sensor Integrated Into Silicon Microfluidic Channel to Prevent Biofouling

To prevent biofouling that typically limits the longevity of electrochemical sensors, presented herein is a cyclic voltammetry sensor that is embedded within a 5 µm radius microfluidic channel microfabricated in a silicon chip. Not only are the electrodes made hidden from the direct biofouling attack in the tissue, but also a thin-layer flow cell configuration provided a 4× increase in faradaic peak current due to a constant supply of analytes.

Implantable in-vivo electrochemical sensors have high spatial, temporal, and chemical resolution. However, being in direct contact with aggressive fouling agents in biological tissue, they often suffer from electrode degradation that limits their lifetime [1]. Microdialysis is an alternative method for long-term in-vivo monitoring of biochemistry, however with significantly lower temporal and spatial resolution. Embedding electrochemical cells inside microdialysis microfluidic channels can potentially protect their electrodes from direct biofoulant attack, thus increasing their longevity while providing a constant supply of analytes in a dialysate flow. However, typical flow rates in microdialysis are fast (µL/min) and microfluidic channel cross-sections are large (>10000 µm²), such that only a fraction of analytes will react at the electrode surface thus drastically decreasing sensitivity. Here we demonstrate an electrochemical cell integrated inside a miniaturized silicon microfluidic chip with scaled 5-µm radius channels that operates in a thin layer regime at ultra-slow nL/min flow rates and exhibits enhanced chemical sensitivity.

To fabricate electrodes integrated inside a silicon microfluidic channel (FIG. 2A), Ti/Pt was deposited on top of a lithographically patterned silicon nitride (SiN) hard mask layer. Microfluidic channels are formed by isotropic etching of silicon substrate through a series of small 1 µm² holes that are then sealed by deposition of a SiN capping layer. Close examination (FIG. 2B) reveals Ti/Pt interdigitated electrodes with 5-µm wide detection sites exposed to in-channel microfluidic flow (bottom, FIG. 2B). To ensure efficient electrolysis in a thin-layer regime [2], the channel radius was designed to be just 5 µm, less than the diffusion-layer thicknesses (~5-50 µm) expected for fast scan cyclic voltammetry (FSCV) scan rates (1 V/s - 100 V/s, 0.06-6 s per scan scanning -3 to +3 V). The resulting packaged in-channel electrochemical sensor (FIG. 2D) is a 2-electrode system with Ti/Pt as the surface metal for both working (WE) and pseudoreference/counter (RE/CE) electrodes.

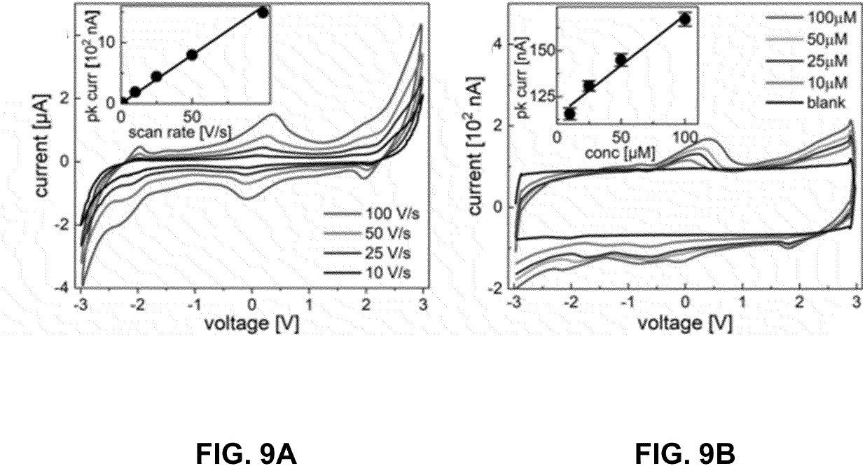

First, CV measurements are performed without active liquid flow when the channels are filled with 1× phosphate buffered saline (PBS) solution and 1 mM aqueous redox probe ferrocene-methanol (FcMeOH). An increase of the scan rate (FIG. 9A) results in a linear increase (inset in FIG. 9A) of the peak current, indicative of a mass transfer-limited electrolysis process within a thin-layer cell [2]. To determine sensitivity, CV measurements were repeated at a constant 100 V/s scan rate for several analyte concentrations (FIG. 9B). Scans reveal an Fc/Fc⁺ oxidative peak potential between 0-0.5 V, similar to potentials reported for standard reference electrode materials. Linear regression (inset in FIG. 9B) of the measured peak currents enables estimation of the limits of detection as 15 µM.

Next, CV measurements are repeated for various flow rates at a fixed 100 V/s scan rate (FIG. 6A). For all tested flow rates, from 10 nL/min up to 100 nL/min, the linear increase of the peak current with the scan rate is well-preserved (FIG. 6B), indicating a stable thin-layer electrolysis regime. The addition of in-channel flow results in a larger peak current than in the no-flow case (FIG. 6B), however this enhancement is not monotonic. For a given scan rate the peak current first increases up to 4× the no-flow case at a flow rate of 50 nL/min (FIG. 6C) and then decreases at faster flows.

Such enhancement of the peak current (hence sensitivity) with introduction of the analyte flow is likely due to the efficient replenishment of analyte molecules in the electrode vicinity during electrolysis that compensates local analyte depletion characteristic to the no-flow case. The subsequent peak current decrease at larger flow rates is likely due to a competition between the analyte residence time at the electrode (~3 ms at 100 nL/min) and a characteristic time of the thin-layer electrolysis (~6 ms).

We have developed an in-channel electrochemical sensor that is monolithically integrated within a silicon-based microfluidic chip to reduce biofoulant exposure to electrode surfaces. The electrochemical cell is designed to operate in a thin-layer regime that is confirmed by linear dependence of oxidative peak current on scan rate. Introduction of 50 nL/min in-channel liquid flow resulted in a 4× increase in sensitivity likely due to an efficient supply of analytes to the electrode vicinity. This enhancement worsened with the increase of flow rate, when the replenishing analyte molecules began to be swept away from the electrode vicinity faster than they could undergo electrolysis. Our results indicate that in-channel electrochemistry embedded in miniaturized microdialysis systems is promising for biofouling prevention and increasing sensor longevity in long-term chronic in-vivo studies.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. Specific names of components are intended to be exemplary, as it is known that one of ordinary skill in the art can name the same components differently.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant’s invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### References for the Examples

(1) Agnati, L. F.; Leo, G.; Zanardi, A.; Genedani, S.; Rivera, A.; Fuxe, K.; Guidolin, D. In Acta Physiologica; John Wiley & Sons, Ltd, 2006; Vol. 187, pp 329-344.
(2) Sykova, E.; Nicholson, C. Physiological Reviews. NIH Public Access October 2008, pp 1277-1340.
(3) Xie, L.; Kang, H.; Xu, Q.; Chen, M. J.; Liao, Y.; Thiyagarajan, M.; O’Donnell, J.; Christensen, D. J.; Nicholson, C.; Iliff, J. J.; Takano, T.; Deane, R.; Nedergaard, M. Science (80-. ). 2013, 342 (6156), 373-377.
(4) Lee, K. H.; Lujan, J. L.; Trevathan, J. K.; Ross, E. K.; Bartoletta, J. J.; Park, H. O.; Paek, S. B.; Nicolai, E. N.; Lee, J. H.; Min, H. K.; Kimble, C. J.; Blaha, C. D.; Bennet, K. E. Sci. Rep. 2017, 7, 1-14.
(5) Bucher, E. S.; Wightman, R. M. Annu. Rev. Anal. Chem. 2015, 8 (1), 239-261.
(6) Ou, Y.; Buchanan, A. M.; Witt, C. E.; Hashemi, P. Analytical Methods. Royal Society of Chemistry Jun. 6, 2019, pp 2738-2755.
(7) Ngernsutivorakul, T.; White, T. S.; Kennedy, R. T. ChemPhysChem 2018, 19 (10), 1128-1142.
(8) Puthongkham, P.; Venton, B. J. Analyst. Royal Society of Chemistry Feb. 21, 2020, pp 1087-1102.
(9) Roberts, J. G.; Sombers, L. A. Analytical Chemistry. American Chemical Society Jan. 2, 2018, pp 490-504.
(10) Lugo-Morales, L. Z.; Loziuk, P. L.; Corder, A. K.; Toups, J. V.; Roberts, J. G.; McCaffrey, K. A.; Sombers, L. A. Anal. Chem. 2013, 85 (18), 8780-8786.
(11) Weber, W.; Fussenegger, M. Nat. Rev. Genet. 2012, 13 (1), 21-35.
(12) Liao, W.; Cui, X. T. Biosens. Bioelectron. 2007, 23 (2), 218-224.
(13) El-Schich, Z.; Zhang, Y.; Feith, M.; Beyer, S.; Sternbaek, L.; Ohlsson, L.; Stollenwerk, M.; Wingren, A. G. Biotechniques 2020, btn--2020--0091.
(14) Bhattacharyya, I. M.; Shalev, G. ACS Sensors 2020, 5 (1), 154-161.
(15) Nakatsuka, N.; Yang, K.-A.; Abendroth, J. M.; Cheung, K.; Xu, X.; Yang, H.; Zhao, C.; Zhu, B.; Rim, Y. S.; Yang, Y.; Weiss, P. S.; Stojanovic, M. N.; Andrews, A. M. Science (80-. ). 2018, 6750 (September), eaao6750.
(16) Cheung, K. M.; Yang, K. A.; Nakatsuka, N.; Zhao, C.; Ye, M.; Jung, M. E.; Yang, H.; Weiss, P. S.; Stojanovic, M. N.; Andrews, A. M. ACS Sensors 2019, 4 (12), 3308-3317.
(17) Lecomte, A.; Descamps, E.; Bergaud, C. J. Neural Eng. 2018, 15 (3).
(18) Kozai, T. D. Y. Y.; Jaquins-Gerstl, A. S.; Vazquez, A. L.; Michael, A. C.; Cui, X. T. ACS Chem. Neurosci. 2015, 6 (1), 48-67.
(19) Dai, X.; Hong, G.; Gao, T.; Lieber, C. M. 2018.
(20) Scaini, D.; Ballerini, L. Curr. Opin. Neurobiol. 2018, 50, 50-55.
(21) Harris, J. P.; Tyler, D. J. Crit. Rev. Biomed. Eng. 2013, 41 (6), 435-456.
(22) Kook, G.; Lee, S. W.; Lee, H. J. H. C.; Cho, I. J.; Lee, H. J. H. C. Micromachines 2016, 7 (10), 1-20.
(23) Wellman, S. M.; Eles, J. R.; Ludwig, K. A.; Seymour, J. P.; Michelson, N. J.; McFadden, W. E.; Vazquez, A. L.; Kozai, T. D. Y. Y. Adv. Funct. Mater. 2018, 28 (12), 1-38.
(24) Schwerdt, H. N.; Shimazu, H.; Amemori, K. ichi; Amemori, S.; Tierney, P. L.; Gibson, D. J.; Hong, S.; Yoshida, T.; Langer, R.; Cima, M. J.; Graybiel, A. M. Proc. Natl. Acad. Sci. U. S. A. 2017, 114 (50), 13260-13265.
(25) Schwerdt, H. N.; Zhang, E.; Kim, M. J.; Yoshida, T.; Stanwicks, L.; Amemori, S.; Dagdeviren, H. E.; Langer, R.; Cima, M. J.; Graybiel, A. M. Commun. Biol. 2018, 1 (1).
(26) Meunier, C. J.; McCarty, G. S.; Sombers, L. A. Anal. Chem. 2019, 91 (11), 7319-7327.
(27) Atcherley, C. W.; Laude, N. D.; Parent, K. L.; Heien, M. L. Langmuir 2013, 29 (48), 14885-14892.
(28) Venton, B. J.; Cao, Q. Analyst. Royal Society of Chemistry Feb. 21, 2020, pp 1158-1168.
(29) Rodeberg, N. T.; Sandberg, S. G.; Johnson, J. A.; Phillips, P. E. M. M.; Wightman, R. M. ACS Chemical Neuroscience. NIH Public Access 2017, pp 221-234.
(30) Takmakov, P.; McKinney, C. J.; Carelli, R. M.; Wightman, R. M. Rev. Sci. Instrum. 2011, 82 (7), 74302.
(31) Howell, J. O.; Kuhr, W. G.; Ensman, R. E.; Mark Wightman, R. J. Electroanal. Chem. 1986, 209 (1), 77-90.
(32) Geladi, P.; Kowalski, B. R. Anal. Chim. Acta 1986, 185 (C), 1-17.
(33) Keithley, R. B.; Mark Wightman, R.; Heien, M. L. TrAC - Trends in Analytical Chemistry. Elsevier 2009, pp 1127-1136.
(34) Ngernsutivorakul, T.; Steyer, D. J.; Valenta, A. C.; Kennedy, R. T. Anal. Chem. 2018, 90 (18), 10943-10950.
(35) Zestos, A. G.; Kennedy, R. T. AAPS J. 2017, 19 (5), 1284-1293.
(36) Lee, W. H.; Ngernsutivorakul, T.; Mabrouk, O. S.; Wong, J. M. T.; Dugan, C. E.; Pappas, S. S.; Yoon, H. J.; Kennedy, R. T. Anal. Chem. 2016, 88 (2), 1230-1237.
(37) Petit-Pierre, G.; Colin, P.; Laurer, E.; Deglon, J.; Bertsch, A.; Thomas, A.; Schneider, B. L.; Renaud, P. Nat. Commun. 2017, 8 (1).
(38) Gu, H.; Varner, E. L.; Groskreutz, S. R.; Michael, A. C.; Weber, S. G. Anal. Chem. 2015, 87 (12), 6088-6094.
(39) Xu, C.; Wu, F.; Yu, P.; Mao, L. ACS Sensors. American Chemical Society Dec. 27, 2019, pp 3102-3118.
(40) Zhang, Y.; Esters, A.; Bi, O.; Vlasov, Y. 2019 20th Int. Conf. Solid-State Sensors, Actuators Microsystems Eurosensors XXXIII (TRANSDUCERS EUROSENSORS XXXIII) 2019, No. June, 209-212.
(41) Zhang, Y.; Shi, W.; Kim, S.; Brenden, C. K. In MicroTAS; 2020.
(42) Lee, W. H.; Slaney, T. R.; Hower, R. W.; Kennedy, R. T. Anal. Chem. 2013, 85 (8), 3828-3831.
(43) Van Den Brink, F. T. G.; Phisonkunkasem, T.; Asthana, A.; Bomer, J. G.; Van Den Maagdenberg, A. M. J. M.; Tolner, E. A.; Odijk, M. Lab Chip 2019, 19 (8), 1332-1343.
(44) Oh, K. W.; Lee, K.; Ahn, B.; Furlani, E. P. Lab on a Chip. Royal Society of Chemistry Feb. 7, 2012, pp 515-545.
(45) Bard, A.J.; Faulkner, L. R. Electrochemical Methods Fundamentals of Electrochemistry, 2001.

TABLES

TABLE 1

Calculated pump parameters for various modes

| Mode | P1 mbar | P2 mbar | Specific Constraints |
|---|---|---|---|
| Sampling | -119 | 0 | Flow from sampling area to detection channel with minimum contribution from bypass channel |
| Transient | 0 | -92 | Flow from sampling area to bypass, detection channel de-pressurized for chemical change |
| Calibration/ Regeneration | 118 | -183 | Flow from sampling area to bypass, flow from detection channel to bypass |
| Rinse | 237 | -275 | Same as calibration/regeneration, with doubled flow in detection channel |

We claim:

1. An electrochemical sensor for detecting an analyte comprising:
a microfluidic channel having a lumen surface extending between a proximal end and a distal end to define a microfluidic lumen;
a microelectrode that forms a portion of the lumen surface and configured for fluid contact with a fluid sample that flows in the microfluidic lumen to detect the analyte in the fluid sample;
a sampling port fluidically connected to the microfluidic channel distal end configured to introduce the fluid sample from a sampling area adjacent to the sampling port to the microelectrode of the microfluidic lumen;
a reagent channel fluidically connected to the microfluidic channel, wherein the reagent channel is configured to introduce a reagent solution to the microelectrode for microelectrode calibration and/or cleaning;
a flow controller fluidically connected to the microfluidic channel and/or the reagent channel to control flow of the fluid sample through the sampling port and the microfluidic channel;
wherein the microfluidic channel, sampling port and reagent channel is formed from a unitary substrate, including a silicon (Si) substrate, wherein the microfluidic lumen has an effective radius as small as 4 µm and capable of withstanding a high pressure during fluid flow, such as a pressure of up to 4 atmospheres, without leakage.

2. The electrochemical sensor of claim 1, comprising a plurality of microelectrodes for multiplex detection of a plurality of analytes from the fluid sample introduced to the microfluidic channel.

3. The electrochemical sensor of claim 1, wherein the reagent solution from the reagent microchannel is introduced to the microfluidic channel to reduce microelectrode fouling associated with a fouling material from the environment surrounding the electrochemical sensor, wherein the reagent fluid does not exit the sampling port into the sampling area.

4. The electrochemical sensor of claim 1, wherein a characteristic dimension of the microfluidic channel cross-section is less than a diffusion layer formed by the fluid sample undergoing laminar flow in the microfluidic channel.

5. The electrochemical sensor of claim 1, wherein the microelectrode is one or more thin film electrodes having a thickness less than 1 µm and a total fluid contact surface area of between 100 µm² and 100 mm².

6. The electrochemical sensor of claim 1, wherein the microelectrode is one or more of:
a functionalized electrode comprising an analyte-specific recognition element, such as a polypeptide, a polynucleotide, an antibody, a molecular imprinted polymer (MIP),
a carbon-fiber electrode;
a parylene-C passivated electrode;
a pyrolyzed photoresist;
a gold electrode;
a platinum electrode;
an ion-selective electrode (e.g., Ag/AgCl);
a boron-doped diamond electrode; and
a titanium electrode.

7. The electrochemical sensor of claim 1, wherein the microelectrode is part of a field-effect transistor (FET).

8. The electrochemical sensor of claim 1, having a form factor configured for implantation into a living animal or person and the analyte is from a biological sample, and the microelectrode positioned in the microfluidic channel resists fouling, thereby increasing operational lifetime of the implanted electrochemical sensor compared to an electrochemical sensor that is not embedded in a microfluidic channel.

9. The electrochemical sensor of claim 1, wherein the reagent solution is selected from the group consisting of:
a calibration solution;
a cleaning solution;
an activating solution; and
an electrode regeneration solution.

10. The electrochemical sensor of claim 1, wherein the reagent microchannel is configured to convey a regeneration solution the electrode for regenerating an electrode surface parameter without disturbing tissue surrounding the implanted electrochemical sensor.

11. The electrochemical sensor of claim 1, having a measurement run time of at least 90 seconds before introduction of the reagent solution to the microelectrode to provide a reset of the measurement run time, without disturbing tissue surrounding the implanted electrochemical sensor.

12. The electrochemical sensor of claim 1, further comprising a membrane positioned upstream of the microfluidic channel for membrane microdialysis.

13. The electrochemical sensor of claim 1, having an implanted cross-sectional area that does not adversely impact surrounding tissue, including less than 1 mm²; less than or equal to 1200 µm²; including a probe cross-section corresponding to about 15 µm × 75 µm.

14. The electrochemical sensor of claim 1, having an in-line calibration mode to calibrate the electrode without disturbing an environment surrounding the electrochemical sensor.

15. The electrochemical sensor of claim 1, wherein the reagent channel is connected to the microfluidic channel to form a microfluidic junction positioned between the sampling port and the microelectrode.

16. The electrochemical sensor of claim 1, further comprising one or more flow controllers operably connected to:
the microfluidic channel proximal end; and/or
a proximal end of the reagent channel;
wherein the flow controller is a variable pressure pump to control pressure and corresponding flow-rate and flow direction in each of the microfluidic channel and reagent channel, optionally without any flow valves, wherein the variable pressure pump generates a fluid sample flow rate through the microfluidic channel that is between 1 nL/min and 300 nL/min.

17. The electrochemical sensor of claim 1, wherein the flow controllers are configured to provide a plurality of electrochemical sensor modes, the modes comprising:
a sampling mode;
a calibration mode;
a regeneration mode;
a cleaning (rinse) mode; and
a transient mode.

18. The electrochemical sensor of claim 1, configured for use as:
an in-brain sensor;
a continuous glucose sensor;
an oxygen reduction sensor in a fuel cell;
a water quality sensor;
a toxin detector;
a corrosion sensor;
a scanning electrochemical microscopy probe;
a pH sensor;
an impedance sensor;
a component of a battery, or
a component of a desalination device.

19. The electrochemical sensor of claim 1, wherein the sampling port is positioned in an insertable portion having a needle geometry, and the sensor is positioned in a non-insertable potion, and the average thickness of the insertable potion is less than the average thickness of the non-insertable portion.

20. A method of making a monolithic in-line electrochemical sensor, the method comprising the steps of:
a) patterning a layer of SiN on a Si substrate, wherein pattern openings in the SiN layer correspond to microelectrode electrode positions;
b) depositing a metal layer over the patterned layer of SiN and exposed Si substrate corresponding to the microelectrode positions;
c) forming a microfluidic network of channels in the Si substrate by buried channel definition using passivation, etch-hole definition and etchant introduction; and
d) plasma etching to form contact pads in electrical contact with the metal layer electrodes adjacent to a portion of the channel;
e) deep Si etching to define an overall geometrical shape of the in-line electrochemical sensor, including a square base with a needle to receive a fluid sample;
f) providing fluid connectors to the microfluidic network of channels for fluid flow control in the channels;
thereby making the monolithic in-line electrochemical sensor.

21. The method of claim 20, wherein the microfluidic network of channels comprises a microfluidic channel with the microelectrodes having a cross-sectional shape that is semicircular with a radius of less than or equal to 20 µm.

22. A method of detecting an analyte, the method comprising the steps of:
inserting the electrochemical sensor of claim 1 into a subject at an implantation site;
introducing a biological sample from the subject to the microfluidic channel via the sampling port;
energizing the microelectrode and detecting an electrical output from the microelectrode;
detecting the presence or absence of the analyte based on the electrical output from the microelectrode, thereby detecting the analyte.

23. The method of claim 22, wherein the biological sample flows through the microfluidic channel at a flow-rate that is less than or equal to 50 nL/min.

24. The method of claim 22, further comprising the step of:
controlling a relative pressure between the reagent channel and the microelectrode channel to flow the reagent fluid through the microelectrode channel;
wherein the reagent fluid is a cleaning solution or a calibration solution, thereby increasing an operational lifetime of the implanted electrochemical sensor to one hour or greater without having to remove the electrochemical sensor from the implantation site.
